(19) United States
(12) Patent Application Publication
(10) Pub. No.: US 2019/0209038 A1
(43) Pub. Date: Jul. 11, 2019

(54) IN-EAR EEG DEVICE AND BRAIN-COMPUTER INTERFACES

(71) Applicant: HOLLAND BLOORVIEW KIDS REHABILITATION HOSPITAL, Toronto (CA)

(72) Inventors: Rami SAAB, Toronto (CA); Thomas Tak Kin CHAU, Toronto (CA)

(21) Appl. No.: 16/242,478

(22) Filed: Jan. 8, 2019

Related U.S. Application Data
(60) Provisional application No. 62/615,108, filed on Jan. 9, 2018.

(51) Int. Cl. A61B 5/0478; A61B 5/04; A61B 5/00; A61B 5/0484

(52) U.S. Cl. CPC A61B 5/0478; A61B 5/04012; A61B 5/6815; A61B 5/048; A61B 5/7267; A61B 5/04842; A61B 5/04845

(57) ABSTRACT

An in-ear EEG device is provided. The in-ear EEG device comprises an over-ear support arm coupled to an enclosure, and an earpiece coupled to the enclosure. The enclosure has a power switch, an analog output, a power input, and a processor. The processor is configured to receive EEG data and generate output data for the analog output. The earpiece collects the EEG data and transmits the EEG data to the processor.

### FIELD

The present disclosure generally relates to the field of brain-computer interfaces and electroencephalogram (EEG) devices.

### INTRODUCTION

In-ear electroencephalography (EEG) is a method for measuring electrical signals from the brain. This technology is garnering increased interest in the research community and more broadly due to its advantages over conventional measurement systems. EEG caps, often used in brain-computer interface (BCI) systems and neuroscience research, present a non-invasive means to collect neural activity. However, difficulty reducing electrode impedances, long setup times, patient discomfort, and limited ability for long-term recording continue to plague EEG measurement systems.

### SUMMARY

In accordance with one aspect, there is provided an in-ear electroencephalography (EEG) device. The EEG device comprises an enclosure, an earpiece coupled to the enclosure, and an over-ear support arm coupled to an enclosure. The enclosure has a power switch, an analog output, a power input, and a processor. The processor is configured to receive EEG data and generate output data for the analog output. The earpiece has two electrodes to collect the EEG data. The earpiece transmits the EEG data to the processor. The over-ear support arm has a reference electrode to collect the EEG data. The over-ear support arm transmits the EEG data to the processor.

In accordance with another aspect, there is provided an in-ear EEG device. The EEG device comprises an over-ear support arm coupled to an enclosure, an analog output, and a power input. The enclosure comprises a printed circuit board (PCB) of the device and includes a processor and a header. The processor is configured to receive EEG data and generate output data. The header is used for connecting an earpiece to the EEG device.

In accordance with another aspect, there is provided a method of validating an in-ear EEG device for use as a brain-computer interface (BCI). The method comprises performing a set of trial experiments on a plurality of subjects where each subject wearing the in-ear EEG device and a clinical EEG cap (e.g., EEG system), extracting P300 features from signals received from the in-ear EEG device, extracting P300 features from signals received from the clinical EEG cap, extracting auditory steady-state response (ASSR) features from the signals received from the in-ear EEG device, extracting ASSR features from the signals received from the clinical EEG cap, classifying the P300 features and ASSR features received from the in-ear EEG device signals, classifying the P300 features and ASSR features received from the clinical EEG cap signals, and comparing the in-ear EEG classifications and the clinical EEG cap signal classifications.

In accordance with another aspect, there is provided a non-transitory computer-readable storage medium comprising computer-executable instructions for validating an in-ear EEG device for use as a brain-computer interface (BCI). The computer-executable instructions cause a processor to extract P300 features from signals received from an in-ear EEG device, extract P300 features from signals received from a clinical EEG cap, extract auditory steady-state response (ASSR) features from the signals received from the in-ear EEG device, extract ASSR features from the signals received from the clinical EEG cap, classify the P300 features and ASSR features received from the in-ear EEG device signals, classify the P300 features and ASSR features received from the clinical EEG cap signals, and compare the in-ear EEG classifications and the clinical EEG cap signal classifications.

In accordance with another aspect, there is provided an in-ear EEG device. The in-ear EEG device comprises an over-ear support arm coupled to an enclosure, and an earpiece coupled to the enclosure. The enclosure has a power switch, an analog output, a power input, and a processor. The processor is configured to receive EEG data and generate output data for the analog output. The earpiece collects the EEG data and transmits the EEG data to the processor.

In various further aspects, the disclosure provides corresponding systems and devices, and logic structures such as machine-executable coded instruction sets for implementing such systems, devices, and methods.

In this respect, before explaining at least one embodiment in detail, it is to be understood that the embodiments are not limited in application to the details of construction and to the arrangements of the components set forth in the following description or illustrated in the drawings. Also, it is to be understood that the phraseology and terminology employed herein are for the purpose of description, and should not be regarded as limiting.

Many further features and combinations thereof concerning embodiments described herein will appear to those skilled in the art following a reading of the instant disclosure.

### DESCRIPTION OF THE FIGURES

Embodiments will be described, by way of example only, with reference to the attached figures, wherein in the figures:

FIG. 1 illustrates an example of an EEG 10-20 system.

FIG. 2 illustrates an example of an in-ear EEG system design.

FIG. 3 illustrates an example of an alternative in-ear EEG system design.

FIG. 4 illustrates, in a three-dimensional rendering, an example of an in-ear EEG mechanical design, in accordance with some embodiments.

FIG. 5 illustrates different elevation and perspective views of the in-ear EEG system, in accordance with some embodiments.

FIG. 6 illustrates, in a signal flow diagram, an example of a signal pathway 600, in accordance with some embodiments.

FIG. 7 illustrates, in a schematic diagram, an example of the topology of the notch filter, in accordance with some embodiments.

FIG. 8 illustrates, in a schematic diagram, an example of a Sallen-Key topology of the high-pass filter, in accordance with some embodiments.

FIG. 9 illustrates, in a schematic diagram, an example of a Sallen-Key topology of the low-pass filter, in accordance with some embodiments.

FIG. 10 illustrates, in a signal flow diagram, an alternative example of a signal pathway, in accordance with some embodiments.

FIG. 11A illustrates, in a computer-aided design (CAD), an example of a printed circuit board of the PCB, in accordance with some embodiments.

FIG. 11B illustrates, in a computer-aided design (CAD), another example of a printed circuit board of the PCB, in accordance with some embodiments.

FIG. 12 illustrates an example of an experimental setup, in accordance with some embodiments.

FIG. 13 illustrates an example of an experimental protocol, in accordance with some embodiments.

FIG. 14 illustrates an example of a P300 feature extraction process, in accordance with some embodiments.

FIG. 15 illustrates an example of an ASSR feature extraction process, in accordance with some embodiments.

FIG. 16 is a view of an example brain-computer interface (BCI) system, in accordance with some embodiments.

FIG. 17 is a view of an example BCI platform and classification device, in accordance with some embodiments.

FIG. 18 is a view of an example interface application, in accordance with some embodiments.

FIG. 19 illustrates, in a flowchart, an example of a method of validating an in-ear EEG device for use as a BCI, in accordance with some embodiments.

FIG. 20A illustrates, in a display, an example of four directional visual cues, in accordance with some embodiments.

FIG. 20B illustrates an example of a system environment showing a participant wearing electrode sensors watching an output unit, in accordance with some embodiments.

FIG. 20C illustrates another example of a system environment showing the participant wearing the electrode sensors watching the display unit, in accordance with some embodiments.

It is understood that throughout the description and figures, like features are identified by like reference numerals.

### DETAILED DESCRIPTION

The present disclosure relates to in-ear EEG as a measurement system. Its small size provides improved user comfort, especially over long periods of time. The size and location also allows for improved discreetness. The location of the electrodes also provides robustness against eye-blink artifacts (though introduces greater susceptibility to artifacts related to facial muscle movements, i.e., mastication). There may be a limited number of electrodes, which precludes the use of EEG processing techniques such as independent component analysis (ICA). It is desirable to overcome the processing hurdles and relatively limited data.

Embodiments of methods, systems, and apparatus are described through reference to the drawings.

Some embodiments herein relate to in-ear electroencephalography (EEG) devices. The following terms are used in this disclosure:

ASSR: auditory steady-state response.

BCI: brain-computer interface.

CAD: computer-aided design.

CMRR: common-mode rejection ratio.

EEG: electroencephalography.

fc: cut-off frequency.

ISI: inter-stimulus interval.

ITR: information transfer rate.

MMN: mismatch negativity.

PCB: printed-circuit board.

PSD: power-spectral density.

SSVEP: steady-state visually evoked potential.

SWLDA: Step-wise Linear Discriminant Analysis.

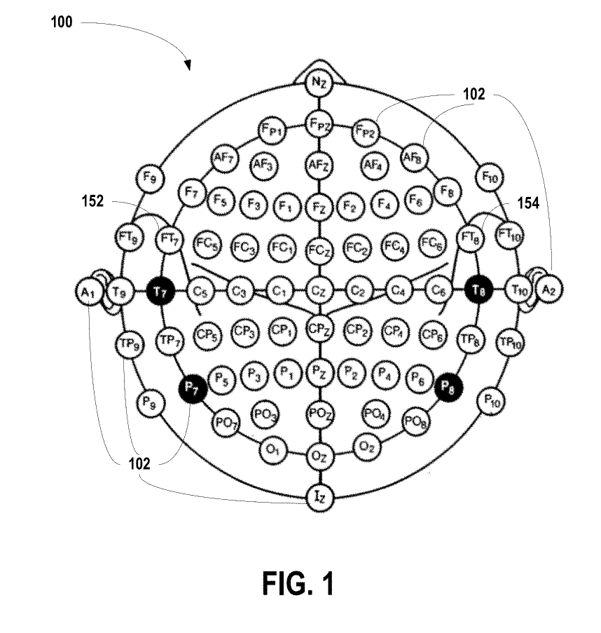

Embodiments described herein relate to brain-computer interfaces and electroencephalogram (EEG) devices. Brain-computer interfaces (BCIs) are a communication pathway between an enhanced or wired brain and an external device. An EEG device detects electrical activity in the brain using electrodes attached to portions of the head. Brain cells communicate via electrical impulses and are active all the time. This electrical activity can be detected and measured by an EEG recording. FIG. 1 illustrates an example of an EEG 10-20 system 100. The figure shows electrode 102 placement and nomenclature as standardized by the American Electroencephalographic Society.

To allow persons with severe motor impairments to communicate, brain-computer interfaces can provide a direct pathway between a user's brain and the outside world. Communicative brain-computer interfaces can be largely grouped into two categories: gaze-independent BCIs and gaze-dependent BCIs. The latter consists of BCI systems which involve direct user control of gaze, and have been the focus of the majority of research related to communicative BCIs. Alternately, gaze-independent BCIs do not require user gaze control, and may be better suited for users with sever motor impairments. Amongst gaze-independent BCIs are those using sound to stimulate users and elicit neural responses; a technique which has shown promise.

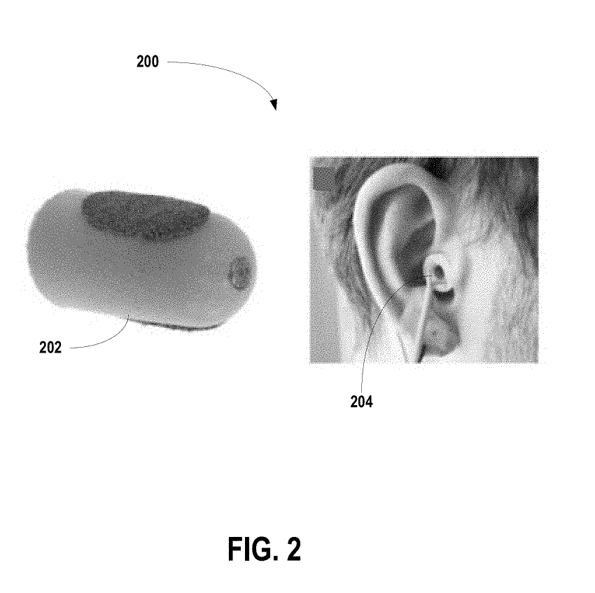

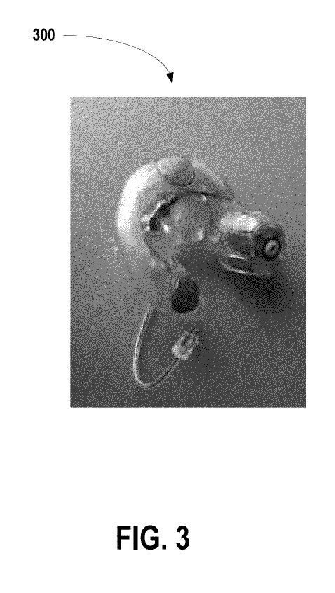

Morphologies of in-ear EEG systems vary between research groups. FIG. 2 illustrates an example of an in-ear EEG system design 200. As shown in FIG. 2, some designs include electrodes implanted into flexible foam ear-plug type substrates 202, while attaching a separate reference electrode to the outside of the earlobe 204. Other designs have simply re-purposed in-ear headphones, used for music listening, into electrical measurement devices. FIG. 3 illustrates an example of an alternative in-ear EEG system design 300 that provides a self-contained earpiece with two electrodes, with the reference extending outside the ear as part of a self-contained package.

The range of BCI paradigms usable with in-ear EEG has not be explored extensively. Nonetheless, there has been early success demonstrated by various groups. Many have shown a notable auditory steady-state response (ASSR). The ASSR is an auditory-evoked neural response to an amplitude or frequency-modulated pure tone; the ASSR is a consequence of the tonotopic organization of the cochlea. The steady-state visually evoked potential (SSVEP), another neural response elicited by a frequency modulated stimulus, can also be measured through in-ear EEG. One research group assessed a three-class SSVEP system with stimulus presented at frequencies of 10, 15 and 20-Hz. Another research has also demonstrated the viability of alpha-attenuation paradigms by asking users to alternate between a math task with their eyes open and resting with their eyes closed.

Groups have also been able to demonstrate the viability of in-ear EEG in detecting other evoked potentials, both auditory and visual. In one study, an auditory odd-ball paradigm was able to elicit a distinct mismatch negativity (MMN) in all 13 subjects. Furthermore, across a series of four sessions the correlation coefficient between all 7200 presented auditory stimuli was 0.80, suggesting consistent recording of the EEG signal using the in-ear system. Correlations were also similar between the ear-lobe referenced signal and the Cz referenced signal; a result corroborated by other research groups and supporting the use of a self-contained in-ear EEG system.

Passive BCIs detect changes in mental-state. Some groups have been able to perform classification between a sub-vocalization task, multiplication task, and rest task with accuracies of up to 70%. As described in U.S. application Ser. No. 15/865,794 (titled “EEG Brain-Computer Interface Platform and Process for Detection of Changes to Mental State” and filed on Jan. 9, 2018, which is hereby incorporated by reference herein in its entirety) in a mental task involving an anagram task, math task, and rest, mental states such as fatigue, frustration and attention were classified with classification accuracies of 74.8%, 71.6% and 84.8%, respectively, using an LDA classifier.

Reactive BCIs use external stimuli in order to elicit specific neural activity. This neural activity is then used to control a communication system. One of the most widely used neural activity in reactive BCIs is the P300 response. This phenomenon was first characterized in 1965 and occurs when an external event is different than that expected and elicits a neural response. Most commonly, this response is produced as part of an odd-ball paradigm, whereby the subject is asked to focus their attention on one of n targets. The targets are presented in a random order. When the desired target is presented, a P300 response is elicited.

Hybrid-BCIs, whereby multiple stimulus modalities are used in conjunction, have boosted information transfer rates (ITRs) when compared to traditional single modality systems. This result has been demonstrated in traditional visual BCI systems. Recently, a group has shown that auditory P300 and ASSR can be combined to improve BCI performance. However, these studies have exclusively used cap-based EEG systems with many electrodes.

The research on relevant BCI protocols for in-ear EEG has been largely concerned with signal quality and whether gross neural signal changes can be detected. There has been limited exploration into the real-world performance of an in-ear EEG BCI in communication applications. In assessing in-ear BCI performance, a reasonable starting point is the confirmation of in-ear automatic detection of well-established P300 and ASSR signals.

Current systems use clinical cap-EEG systems, which, besides being expensive, require long-setup times, and are also impractical for extended periods of use. An EEG system which can be used with existing BCI experimental paradigms while being less expensive, more comfortable, and more discreet, would prove highly useful. To this end, a novel active in-ear EEG system and to test the device using a hybrid P300-ASSR paradigm is desirable.

Though in-ear EEG systems provide a fewer number of electrodes than existing cap-based EEG systems, they have a smaller size and an ability to be discretely worn over longer periods of time. This small size and focus on long-term usability presents some engineering challenges. Specifically, the electrical and mechanical design should be adequately miniaturized to reduce weight and increase user comfort during use. Existing in-ear EEG systems presently exist in research environments and contain passive electrodes. Though the use of passive electrodes simplifies the design, they also reduce overall signal output quality due to the signal transmission over a longer distance before amplification. This leaves the EEG signal (which itself is on the order of micro-volts) highly susceptible to electrical noise. To avoid this, in one embodiment, active filters directly within the earpiece are implemented.

In some embodiments, an in-ear EEG system includes an active filter system capable of amplifying and filtering the micro-volt amplitude EEG signal. The distance between the electrode and the filter system in an in-ear EEG system is preferably minimized to less than 1 centimetre (cm).

In some embodiments, the in-ear EEG system may include wireless capabilities such as a Bluetooth, Wi-Fi or other radio for transmitting measurements taken from the in-ear EEG device to a server for processing.

In some embodiments, the in-ear EEG system mechanical design includes a computer-aided design (CAD) of the device enclosure accounting for wearability. The in-ear EEG system electrical design includes active filters and corresponding printed-circuit board (PCB) for eventual miniaturization of the device. In some embodiments, each filter may be on a separate chip. In other embodiments, multiple filters may be on the same chip.

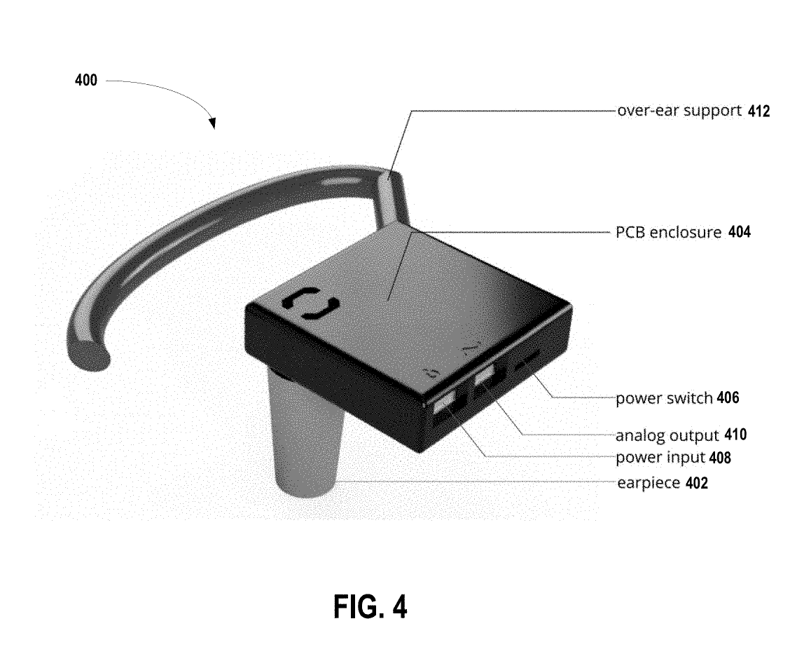

FIG. 4 illustrates, in a three-dimensional rendering, an example of an in-ear EEG device 400, in accordance with some embodiments. The in-ear EEG device 400 comprises an earpiece 402, a PCB enclosure 404, a power switch 406, and connections for both electrical power input 408 and analog output 410. An over-ear support 412 is placed around the ear-lobe so as to ensure proper placement of the device and enhance comfort over long periods of use. Furthermore, a contiguous hole exists in the earpiece 402, PCB and enclosure 404, which should allow for reduced sound attenuation and enable the wearer to hear their environment.

In some embodiments, the over-ear support 412 may be shaped as a hook or other shape to be placed around the earlobe. In other embodiments, the over-ear support 412 may comprise an earlobe clip. In other embodiments, a unit may cover both ears with an earpiece 402 at one or both ears.

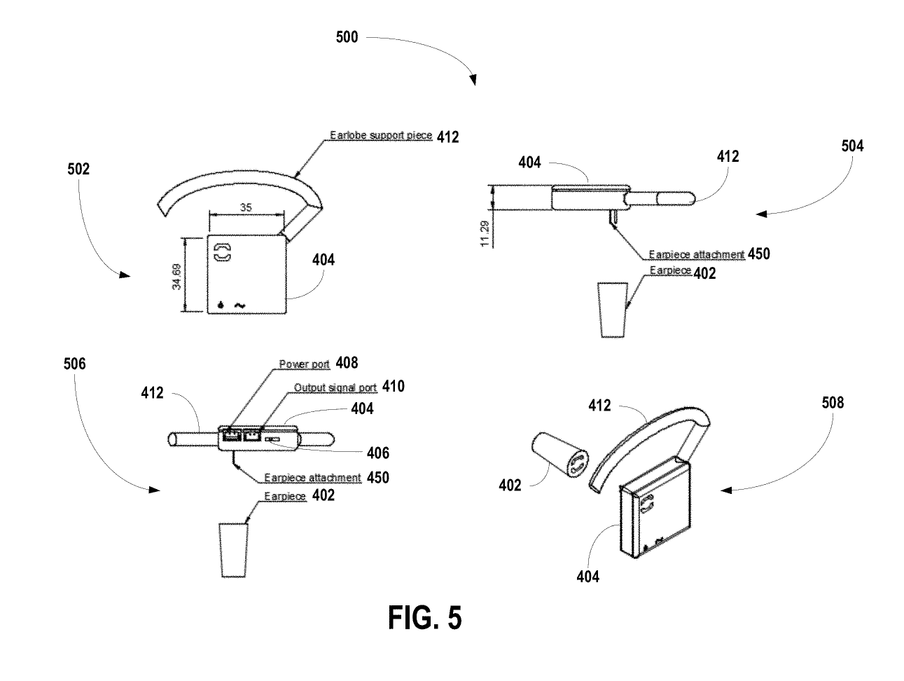

FIG. 5 illustrates different elevation and perspective views 500 of an example of the in-ear EEG system 500, in accordance with some embodiments. In this example, two of the dimensions (length and width) of the PCB enclosure 404 are shown as being 34.69 millimetres (mm) by 35 mm in a first plan view 502. In a second plan view 504, the third dimension (depth) is shown as 11.29 mm. It is understood that the selection of which dimensions are considered as length, width and depth are arbitrary, and that other dimension sizes may be used in other examples. The third plan view 506 shows the power port 508 and output signal port 410. The second 504 and third 506 plan views, and a perspective view 408, show that the earpiece 402 may be detachable from the device 400 and connected to earpiece attachment 450. The device may be manufactured using additive manufacturing (i.e., 3D printing).

The electrical signal produced by neural activity in the brain (the basis of EEG) is on the order of microvolts. Frequencies of interest in the EEG signal include the delta (0.5 to 3.5-Hz), theta (4 to 7-Hz), alpha (8 to 13-Hz), beta (15 to 28-Hz) and gamma (30 to 70-Hz) bands. The small magnitude of the EEG signal necessitates amplification so that its magnitude is adequate to be discretized by an analog-to-digital converter (ADC). Furthermore, environmental noise (namely, 60-Hz electrical power line noise) and aliasing present the need to filter the raw EEG signal prior to digital conversion.

Any time wiring is involved, electrical noise in the environment could inject false signals into the wires before they reach the processor. Buffers (i.e., common buffer amplifiers in analog circuit design) may be added near the electrodes inside the ear allowing for shorter wires between the electrodes and the first piece of circuitry. A buffer amplifier is a circuit that separates the input signal from the downstream electronics (including the length of wiring). It is possible for downstream electronics, and specifically relatively long wires, to affect and change the input signal, and a buffer amplifier separates the two parts of the circuit, by adjusting the effective impedances seen by the input signal and the downstream electronics, so that the effects are minimized.

The buffers may also help minimize the impact of environmental noise. Thus, downstream electronic components (e.g., amplifier, filters, processor, etc.) may be positioned further away from the electrode pads than they otherwise could without the buffers. This, in turn, provides more freedom to the physical design of the system. A design where the downstream components are next to the ear would not need buffers. Buffers could be used when the physical design of the system has those components further away.

An amplifier is a signal stage that increases the signal strength before the signal reaches the processor. Since the processor has a set resolution, a small signal may be too weak to be picked up by the processor, even if it contained valuable information. The amplifier would allow the signal to be stronger, so that the processor can pick up the variations in the signal. Whether or not an amplifier is to be used may depend on the resolution of the processor and the types of signals that are to be recorded from the electrodes.

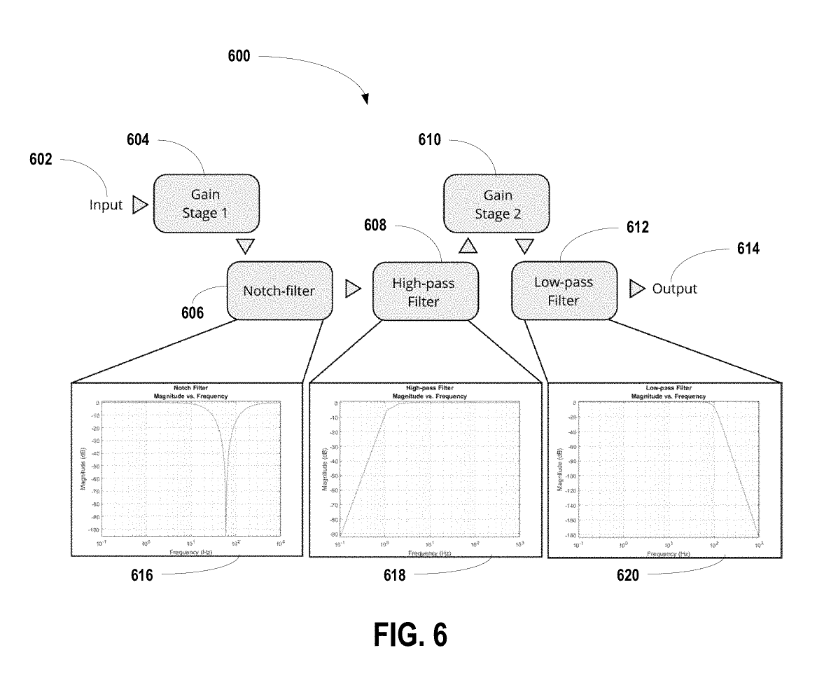

FIG. 6 illustrates, in a signal flow diagram, an example of a signal pathway 600, in accordance with some embodiments. The signal pathway 600 is shown from input 602 to output 614 along with the LTSpice© simulations 616, 618, 620 of the filter magnitude characteristics. This example includes a 60-Hz notch-filter 606 used after a first gain stage 604, a high-pass filter 608 (f_{c}=1-Hz) used prior to a second gain stage 610, and a low-pass filter 612 (f_{c}=100-Hz). The simulations 616, 618, 620 plot magnitude vs. frequency of the signals passing through the filters 606, 608, 612.

In some embodiments, the input 602 may comprise two electrodes 102 placed inside the ear-canal (on the earpiece 402), along with a reference electrode placed either on the earlobe or the mastoid. In the case of the latter design choice, the reference electrode will follow the curve of the ear-lobe support. These electrodes may serve as the positive and negative inputs of the first-stage operational-amplifier, and may be placed approximately 180 degrees apart on the earpiece to maximize the differential signal.

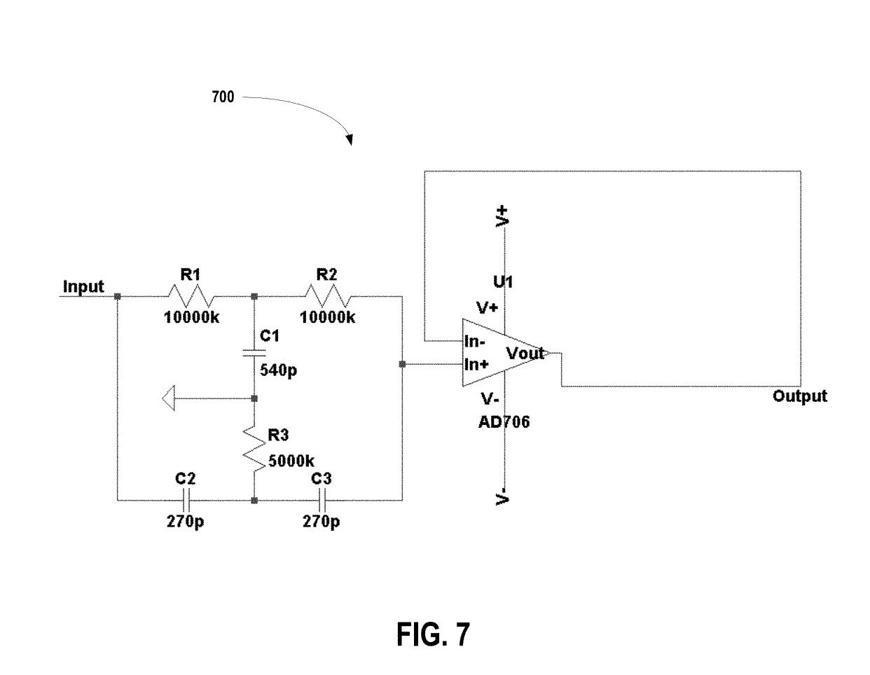

Electrical power-line noise (60-Hz in North-America, and 50-Hz in Europe) can cause undesirable noise in the collected signal even when attempts to mitigate this through the use of differential amplifiers with a high common-mode rejection ratio (CMRR). A notch-filter 606 (otherwise known as a band-reject filter) centered at 60-Hz may be implemented in the signal pathway 600 to reduce the effect of the 60-Hz on the resulting output signal. FIG. 7 illustrates, in a schematic diagram, an example of the topology 700 of the notch filter 606, in accordance with some embodiments.

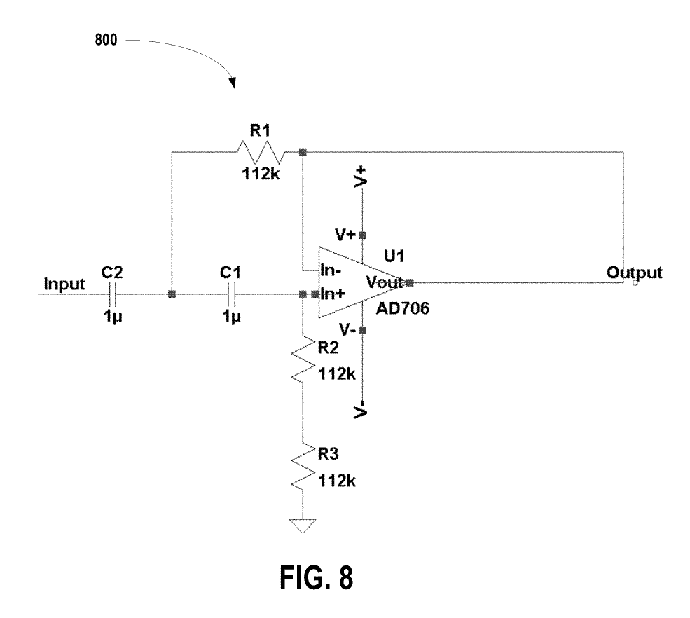

A high-pass filter 608 may be used prior to the second gain stage 610 in order to remove 0-Hz (DC) offset in the signal. This provides that there is minimal to no saturation of the output signal, and provides that information from the signal is not lost. FIG. 8 illustrates, in a schematic diagram, an example of a Sallen-Key topology 800 of the high-pass filter 608, in accordance with some embodiments.

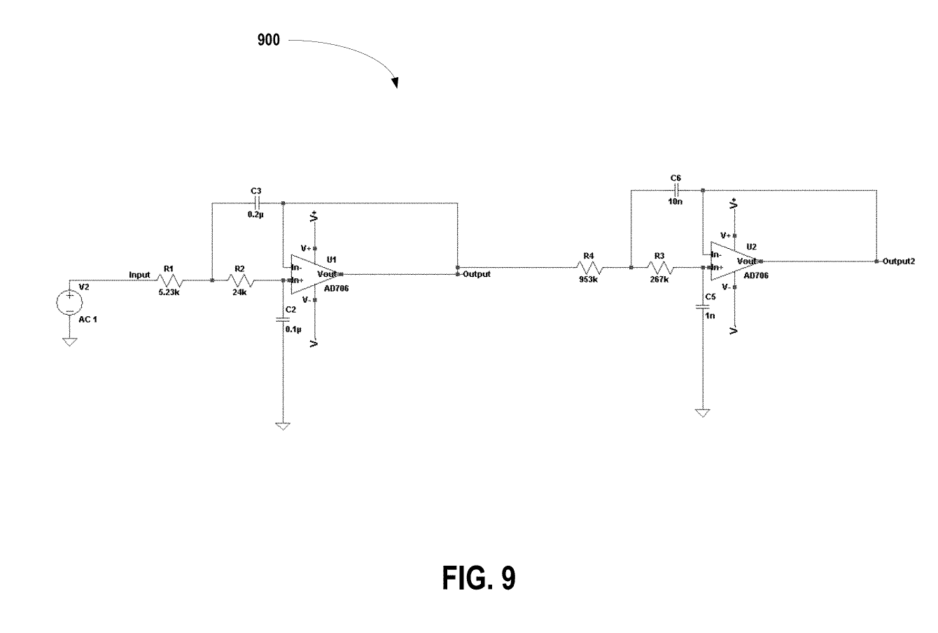

Aliasing, the process whereby digital sampling causes shifts in frequencies above the Nyquist frequency is prevented by low-pass filtering the signal so that there is sufficient attenuation of frequencies above half the sampling frequency. In order to ensure adequate attenuation and to enable the use of lower sampling frequencies, a low-pass Butterworth filter of order 4 was used with a cut-off of (f_{c}=100-Hz). This assumes a sampling frequency of 1000-Hz and will suppress signals above 500-Hz (i.e., fs/2) by approximately −56 dB. FIG. 9 illustrates, in a schematic diagram, an example of a Sallen-Key topology 900 of the low-pass filter 612, in accordance with some embodiments. This topology 900 may be used to realize this analog filter, and the associated component values are shown in FIG. 9.

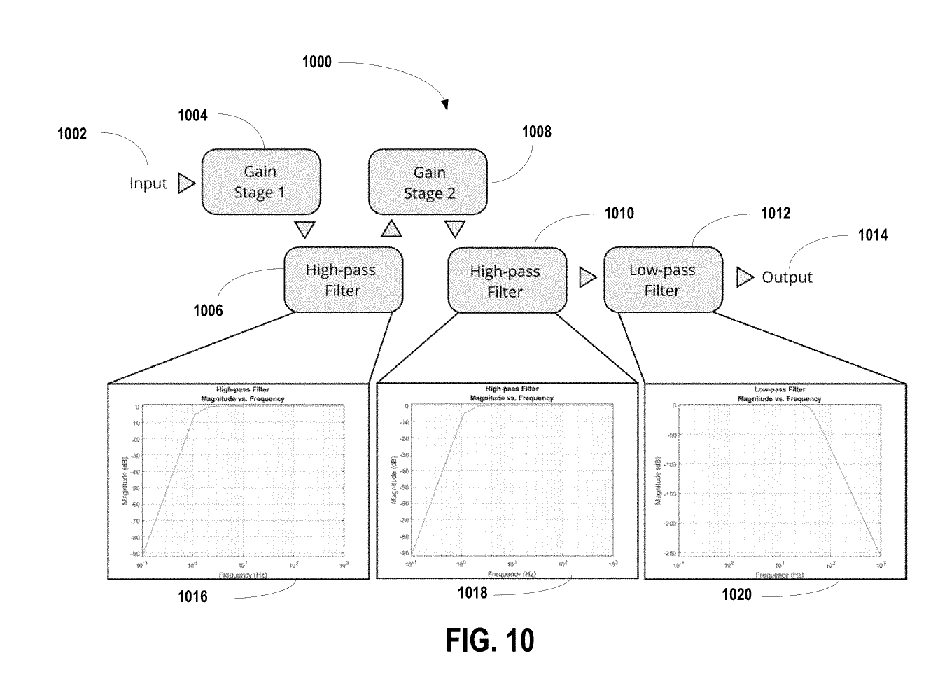

FIG. 10 illustrates, in a signal flow diagram, an alternative example of a signal pathway 1000, in accordance with some embodiments. The signal pathway 1000 is shown from input 1002 to output 1014. The signal pathway 1000 does not include a notch filter. The signal is passed through a high-pass filter 1006 after a first gain stage 1004 and prior to a second gain stage 1008. After the second gain stage 1008, the signal is passed through a high-pass filter 1010 and then to a low-pass filter 1012 with a cut-off frequency of 40-Hz (rather than 100-Hz). An advantage of this alternative design is that it precludes the need for a 60-Hz notch filter. However, the lower cut-off frequency of the low-pass filter 1012 will remove some information-containing EEG components above 40-Hz (specifically in the gamma band). Simulations 1016, 1018, 1020 show filter magnitude characteristics of the high-pass filters 1006, 1010 and low-pass filter 1012. Other combinations of filters and gains are possible in other examples. For example, the signal pathway 1000 may be modified such that the low-pass filter is 100-Hz and an additional gain stage is added.

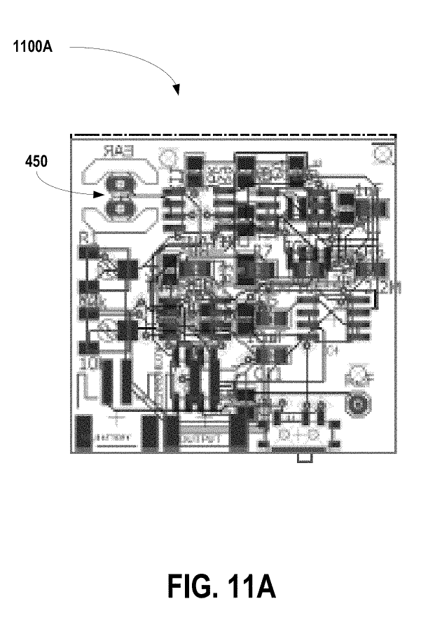

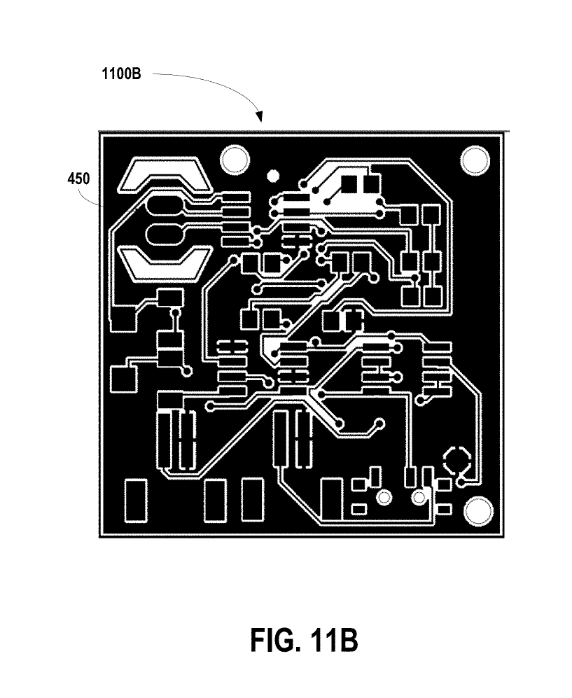

In some embodiments, the PCB will be designed to miniaturize the design and ensure the reliability of the electrical components. FIG. 11A illustrates, in a computer-aided design (CAD), an example of a PCB 1100A, in accordance with some embodiments. To enable customizable gains during the development phase, two variable resistors (potentiometers) may be added to the board 1100A. The 2-pin header 450 to connect to the earpiece 402 is visible in the top-left corner of the board 1100A along with the holes in the PCB 1100A to allow for sound to pass through the device 400. FIG. 11B illustrates, in a CAD, the top layer 1100B of the PCB 1100A. The 2-pin header 450 is also shown in FIG. 11B.

A study may be performed to assess using the in-ear EEG device 400 for a P300-ASSR BCI. For example, a study may determine the level of classification accuracy that can be achieved when deploying an in-ear EEG device 400 measurement system in a P300-ASSR BCI paradigm with typically developed adults. The in-ear EEG device 400 may be used to collect data from patients (or subjects in studies). For example, 15 consenting typically developed adults may be the patients (or subjects). A subject may wear the in-ear EEG device 400.

In one study embodiment, an ASSR task may be used to determine if a signal originates from neurons in the brain, and will be used to assess the fidelity of recording using the in-ear EEG electrode. White noise amplitude-modulated at 37-Hz and 43-Hz may be presented to the user for one minute, and the resulting signal from the in-ear EEG device 400 may be collected. The user may be instructed to focus on the sound, after which the user will rest for 20-seconds and then repeat this process for a total of 20 trials.

The in-ear EEG device 400 may be worn in both ears of patients to collect activity in both hemispheres of the brain. For validation purposes, measurements may also be made simultaneously via a clinical cap-based EEG system. Validating signals may be acquired from electrodes 102 placed at 32 electrode locations of the international 10-20 system 100. The FT7 152 and FT8 154 are added as they are prime candidates for reference electrodes 102 using an in-ear EEG device 400. An illustration of the 10-20 system 100 is shown in FIG. 1. In some study embodiments, subjects may also wear the in-ear EEG device 400 and data may be simultaneously recorded from both the cap and in-ear device. The signal processing, and classification methods which follow will be performed identically on both the gross cap-EEG data as well as the in-ear EEG data.

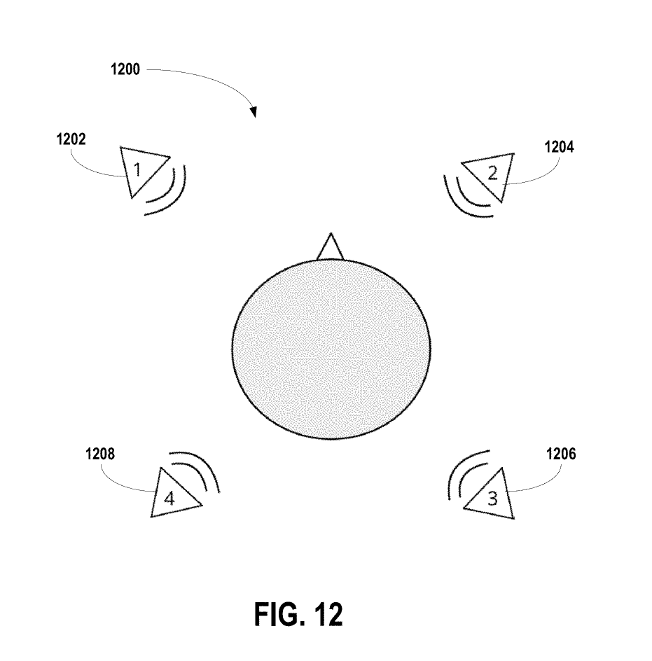

FIG. 12 illustrates an example of an experimental setup 1200, in accordance with some embodiments. Preferably, research subjects will be seated comfortably in-front of a computer monitor. A fixation cross may be presented on the screen for the duration of the experiment to mitigate eye-blink artifacts. Surrounding the subject may be four speakers (1202, 1204, 1206, 1208) each corresponding to a different target (1, 2, 3 and 4) as shown in FIG. 12. Each target may, for the duration of the stimulus period, play Gaussian white noise AM-modulated at 37-Hz, 43-Hz, 46-Hz and 49-Hz respectively.

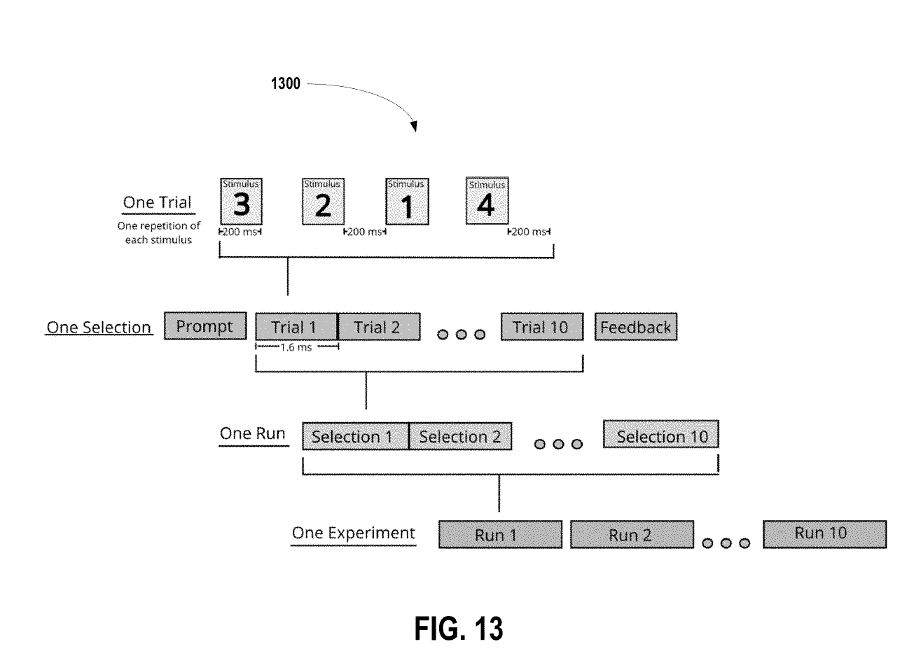

At the start of the experiment, subjects are asked to focus on one of the four speakers 1202, 1204, 1206, 1208. In some embodiments, this prompt may be a computerized voice asking the subject to “Please focus on speaker X” and may come from the corresponding speaker X. This prompt may last for a duration of approximately 1.5 seconds. After this, the stimulus period may begin and the four speakers 1202, 1204, 1206, 1208 may produce the previously described AM-modulated noise. In a pseudo-random order, the volume of the AM-modulated noise from one of the targets (1, 2, 3 or 4) may increase for a duration of 200-ms (milliseconds) followed by 200-ms of equal volume (denoted as the inter-stimulus interval (ISI)), and then another random target may increase in volume relatively sharply for a duration of 200-ms. This process may continue until each target has increased in volume (i.e., a single repetition of each speaker 1202, 1204, 1206, 1208 increasing in volume). FIG. 13 illustrates an example of an overview of an experimental protocol 1300, in accordance with some embodiments. One example trial sequence, 3-2-1-4 is shown. These sequences may be randomly generated for each trial to avoid user adaptation. This single repetition of stimuli may be presented to the subject as part of a single trial block and forms the basic P300 eliciting odd-ball paradigm.

Each experiment may be divided into two phases: a training phase, and an online-spelling phase. During the training phase, the subject may be prompted with a speaker to focus on one of the speakers, and each selection will consist of 10 trial blocks. In some embodiments, no feedback is provided to the user after each selection during the training phase. Each run may comprise 10 selections, and the training phase may comprise 10 runs, totaling 100 selections.

During the online phase, the number of trial blocks may dynamically change based on the confidence of the machine learning algorithm. Each trial is followed by a feedback period whereby a voice prompt is used to convey the target the computer believes the user was focusing on. The online phase may last for a duration of 5 runs (50 selections).

In one study embodiment, 15 participants will be recruited that are 18 years of age or older, have no history of stroke or other neurological conditions, have normal or corrected-to-normal vision, and have normal hearing.

Both P300 and ASSR features may be extracted from the EEG signal. These complimentary features may then be used together in the machine learning algorithm to classify user selections.

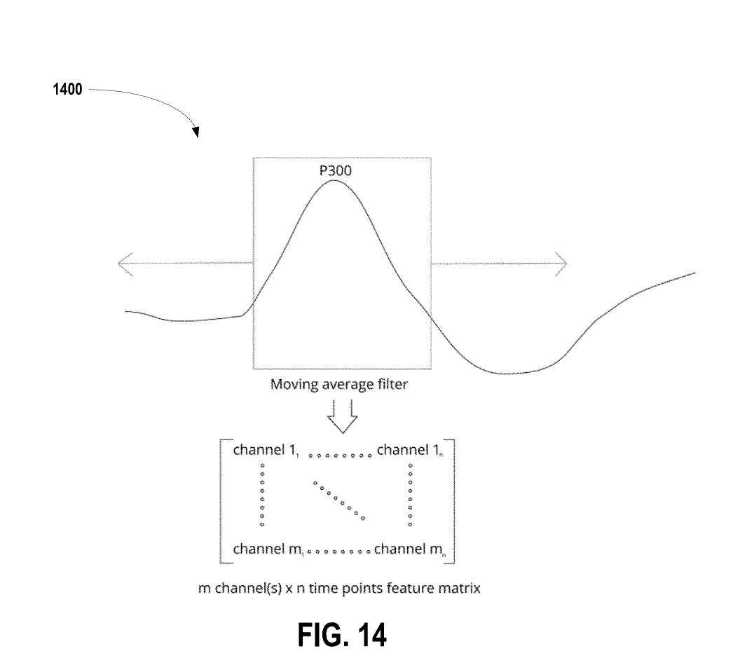

Some groups have used simple decimation to down-sample (i.e., taking every nth sample to reduce the number of data points) to extract P300 features from the EEG signal. However, this method is susceptible to collecting random noise in the data. The P300 features may be extracted by using a moving average filter and taking the average of every 40 samples resulting in a sample rate of 1000-Hz/40=25-Hz. FIG. 14 illustrates an example of a P300 feature extraction process 1400, in accordance with some embodiments. This is equivalent to first low-pass filtering the signal and then retaining every nth sample; a process known as decimation.

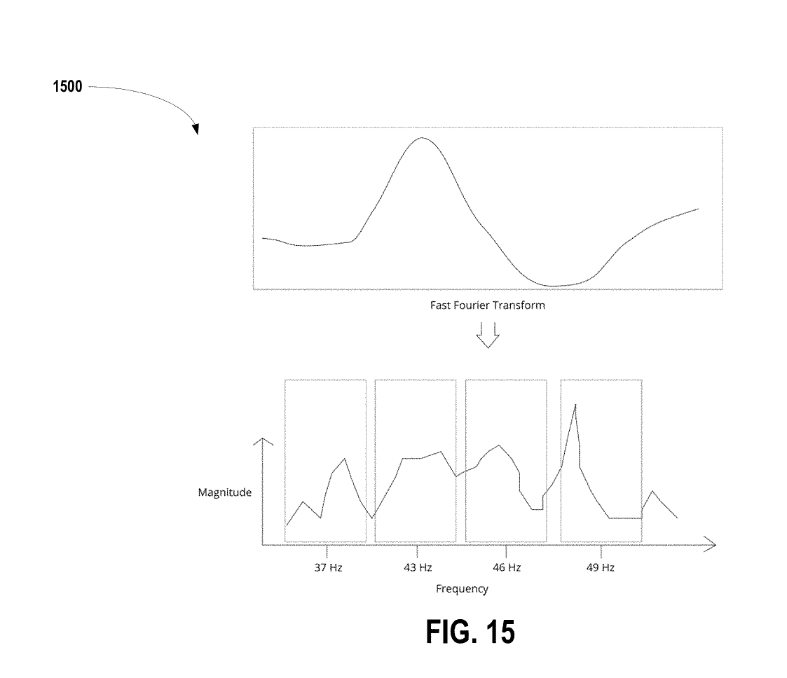

FIG. 15 illustrates an example of an ASSR feature extraction process 1500, in accordance with some embodiments. A fast-fourier transform may be performed on the EEG data and the resulting frequency domain data used to calculate the power-spectral density (PSD) with bins centered at the target frequencies+/−1-Hz. A feature vector including the PSD in each bin for each trial may be used in the machine learning algorithm.

BLDA classifiers, or other machine learning (i.e., neural networks, deep learning, etc.) classifiers, may be used to train both the P300 and ASSR classifiers. Each trial may produce a P300 feature vector along with an ASSR feature vector for both targets and non-targets. These labelled vectors may be used to train two separate Step-wise Linear Discriminant Analysis (SWLDA) classifiers, one for the P300 and one for the ASSR. This will produce an ASSR and P300 score. SWLDA, like other linear-discriminant analysis algorithms, assumes a normal data distribution with equal covariance between class one and two. Its aim is to find a class-separating hyperplane that maximizes the separation of the class means while minimizing inner class variance.

$${Score}^{p 300} = \frac{1}{K} {\sum }_{i = 1}^{K} {Y}_{ij}^{p 300}$$

Here i represents the trial number, j is the target number, K is the total number of trials and Y is the P300 response score calculated using the down-sampled raw EEG data multiplied by the weights determined using the SWLDA classifier.

Similarly, for the ASSR classification, the feature vector may be tagged with either a target or a non-target label using the subject training data. These labeled vectors may then be used to train a SWLDA classifier that will be used to classify new data in the online section of the experiment.

This will produce a Score^{fusion} for each target (i.e., class). The ASSR and P300 scores may be fused as:

Score_{c}^{fusion}=wc1*Score_{c}^{ASSR}+wc2*Score_{c}^{P300}

The SWLDA class with the highest fusion score may be classified as the target class.

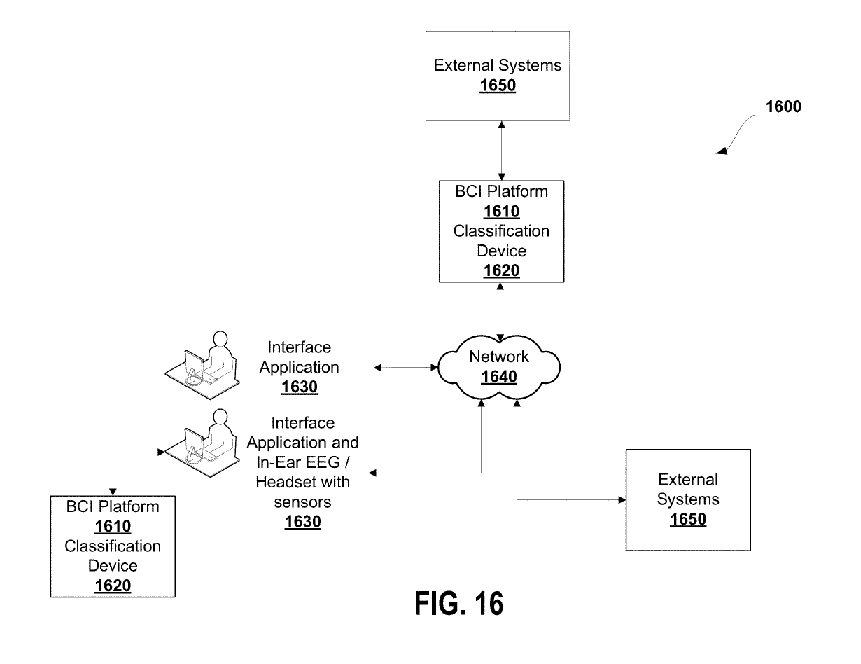

FIG. 16 is a view of an example brain-computer interface (BCI) system 1600, in accordance with some embodiments. BCI system 1600 includes BCI platform 1610, which includes classification device 1620. BCI platform 1610 connects to interface application 1630, for example, to gather EEG data or other data from a user engaged with interface application 1630. The data gathered or a modification of the data gathered may encode communication or input (such as EEG signals or other readings denoting brain activity) from individuals who are performing mental tasks. The interface application 1630 can include electrodes to generate EEG signals. Interface application 1630 can include other sensors, for example. Interface application 1630 and BCI platform 1610 can receive other types of data, including imaging data, for example. Interface application 1630 can include one or more clocks to synchronize data collected from different sensors and modalities.

BCI platform 1610 can connect to interface application 1630 to cause one or more questions to be presented to a user engaged at interface application 1630, and to receive one or more responses to questions or other data input from the user. The questions can be presented on a display device using an interface generated by interface application 1630. The questions can be presented by way of an audio signal and speaker, as another example. BCI platform 1610 can organize the received data or aggregate the data with other data. For example, data from a question and answer exchange with a user can be used by BCI platform 1610 to verify collected EEG data encoding the user's mental state. BCI platform 1610 can organize the received data or aggregate the data with other data using time stamps and clock data for synchronization.

Interface application 1630 can engage a user, for example, via electrodes 102 strategically placed on the user's scalp corresponding to brain regions providing discriminative information or showing task-based activation, such as data corresponding to mental state. In some embodiments, the electrodes 102 may form part of a headset that is engaged with a BCI platform 1610, or houses a BCI platform 1610. The headset can additionally process data. Interface application 1630 can also engage a user via a display, interactive display, keyboard, mouse, or other sensory apparatus. Interface application 130 can transmit and receive signals or data from such devices and cause data to be sent to BCI platform 1610. In some embodiments, the headset may comprise the in-ear EEG device 400 monitoring a subset of the electrodes 52.

In some embodiments, interface application 1630 can process data before sending the data via network 1640 and/or to BCI platform 1610. A user can be engaged with interface application 1630 via electrodes 102, or a headset or in-ear EEG device 400. In some embodiments, BCI platform 1610 and/or classification device 1620 can be housed in the headset or other means of engagement with interface application 1630. In some embodiments, BCI platform 1610 and/or classification device 1620 can connect to interface application 1630 over a network 1640 (or multiple networks).

Classification device 1620 associated with BCI platform 1610 can receive sensor data, for example, EEG data from a single user via interface application 1630. Classification device 1620 can receive stored data from one or more external systems 1650 or interface applications 1630, such as data corresponding to other sessions of data collection, for example. Classification device 1620 can build or train a classification model using this data, for example, EEG data from a single user. Classification device 1620 can use the classifier to classify mental states of the user and cause a result to be sent to an entity (such as external system 1650) or interface application 1630. The result can cause an entity to actuate a response, which can be an alert to a caregiver, or data for a researcher.

The classifier can be re-trained on additional EEG data, for example, data collected from the user at a more contemporaneous time. This may improve the accuracy of the classifier, for example, if same session data are more relevant than data collected from previous days. Further, additional data may improve the accuracy of the classifier so it can be continuously updated and trained as more data and feedback is provided to the BCI platform 1610.

BCI platform 1610 can connect to interface application 1630 via a network 1640 (or multiple networks). Network 1640 (or multiple networks) is capable of carrying data and can involve wired connections, wireless connections, or a combination thereof. Network 1640 may involve different network communication technologies, standards and protocols, for example.

In some embodiments, external systems 1650 can connect to BCI platform 1610 and/or classification device 1620, for example, via network 1640 (or multiple networks). External systems 1650 can be one or more databases or data sources or one or more entities that aggregate or process data. For example, an external system 1650 can be a second BCI platform 1610 that collects EEG data (or other data), performs feature extraction on the data, and builds a classification model. The external system 1650 can then process the data and/or build one or more classification models based on a selection of features. The one or more classification models can be used by one or more other BCI platforms 1610, stored in a database, and/or transmitted to an external system 1650, for example, that is accessible by researchers or developers.

External systems 1650 can receive data from an interface application 1630, BCI platform 1610, and/or classification device 1620. This data can include raw data collected by interface application 1630, such as EEG data from electrodes 102 placed on a user's scalp, data processed by interface application 1630, BCI platform 1610, and/or classification device 1620 (including a classification device 1620 housed in a headset associated with electrodes 102 placed on a user's scalp or in-ear device 400), and/or data from one or more other external systems 1650. This connectivity can facilitate the viewing, manipulation, and/or analysis of the data by a researcher, developer, and/or healthcare provider engaged with an external system 1650.

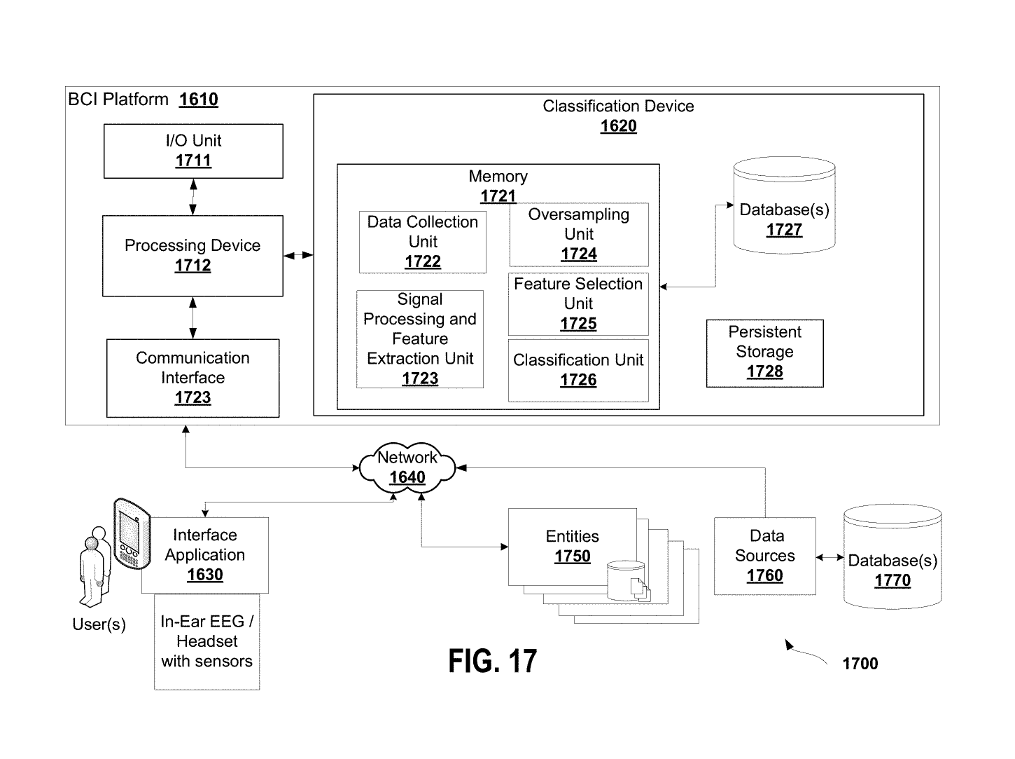

FIG. 17 is a view of an example BCI platform 1610 and classification device 1620, in accordance with some embodiments. A BCI platform 1610 can include an I/O unit 1711, processing device 1712, communication interface 1723, and classification device 1620.

A BCI platform 1610 can connect with one or more interface applications 1630, entities 1750, data sources 1760, and/or databases 1770. This connection may be over a network 1640 (or multiple networks). BCI platform 1610 receives and transmits data from one or more of these via I/O unit 1711. When data is received, I/O unit 1711 transmits the data to processing device 1712.

Each I/O unit 1711 can enable the BCI platform 1610 to interconnect with one or more input devices, such as a keyboard, mouse, camera, touch screen and a microphone, and/or with one or more output devices such as a display screen and a speaker.

A processing device 1712 can execute instructions in memory 1721 to configure classification device 1620, and more particularly, data collection unit 1722, signal processing and feature extraction unit 1723, oversampling unit 1724, feature selection unit 1725, and classification unit 1726. A processing device 1712 can be, for example, any type of general-purpose microprocessor or microcontroller, a digital signal processing (DSP) processor, an integrated circuit, a field programmable gate array (FPGA), a reconfigurable processor, or any combination thereof. The oversampling is optional and in some embodiments there may not be an oversampling unit.

Memory 1721 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Classification device 1620 can include memory 1721, databases 1727, and persistent storage 1728.

Each communication interface 1723 can enable the BCI platform 1610 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

The BCI platform 1610 can be operable to register and authenticate users (using a login, unique identifier, and password for example) prior to providing access to applications, a local network, network resources, other networks and network security devices. The platform 1610 may serve one user or multiple users.

The database(s) 1727 may be configured to store information associated with or created by the classification device 1620. Database(s) 1727 and/or persistent storage 1728 may be provided using various types of storage technologies, such as solid state drives, hard disk drives, flash memory, and may be stored in various formats, such as relational databases, non-relational databases, flat files, spreadsheets, extended markup files, etc.

Classification device 1620 can be used to build a classification model by training on data received from interface application 1630 or other entities 1750, for example, EEG data collected during a change in mental state of a user. Data collection unit 1722 associated with a classification device 1620 and BCI platform 1610 can receive data, for example, EEG data from a single user via interface application 1630. Data collection unit 1722 can receive stored data from one or more external systems (or entities 1750) or interface applications 1630, for example, corresponding to other sessions of data collection.

Signal processing and feature extraction unit 1723 associated with a classification device 1620 can process the data or EEG signals, for example, to remove linear trends, electrical noise, and EEG artifacts, and can reconstruct the EEG signal from the remaining components.

Signal processing and feature extraction unit 1723 can extract features from the data or EEG data using one or more feature extraction methods, such as common spatial pattern, matched-filtering, spectral power estimates, or auto-regressive (Yule-Walker) model of order of magnitude, e.g., three, or wavelet transform. This can produce a vector of features. The order of magnitude can vary.

Oversampling unit 1724 can sample the data or EEG data, for example, to oversample data collected at a more contemporaneous time. In some embodiments, cost-sensitive classification can be used to give the more contemporaneous data larger coefficients in the cost function compared to data collected on, for example, a previous day. Oversampling unit 1724 can thus facilitate higher classification accuracies, for example, by oversampling data collected from the same session that the classification model, once built, will be used to classify EEG data. The oversampling is optional, and in some embodiments there may not be an oversampling step.

Feature selection unit 1725 can select features from the features extracted from the data or EEG data. This may help reduce or avoid overfitting the data, facilitate the generalizability of the data, or facilitate the applicability of a classifier modelled on the data or features extracted from the data. In some embodiments, a classification model is trained on data or features selected from a single user, for example, the ten best features extracted from a set of features extracted from the data collected from the user. The features may be selected based on how they relate to accuracy of the resulting classification model or lowest error.

Classification unit 1726 associated with the classification device 1620 can use the selected features to train an algorithm, such as a linear support vector machine. The algorithm can be used for machine learning classification of data to facilitate classification of mental state given EEG data as input. For example, BCI platform 1610 can use EEG data to build a support vector machine classification model for a particular user who was or is engaged with interface application 1630. The classifier can be re-trained on additional EEG data, for example, data collected from the user at a more contemporaneous time. This may improve the accuracy of the classifier, for example, if same session data are more valuable than data collected from previous days.

At a later time or at a time immediately following re-training of the classifier, interface application 1630 can receive EEG data from the user, for example, corresponding to the user's mental state. Interface application 1630 can transmit the data to BCI platform 1610. As described above, data collection unit 1722 can collect the EEG data, signal processing and feature extraction unit 1723 can process the data and extract features, feature selection unit 1725 can select the relevant subset of features, and classification unit 1726 can use the personalized classification model for that user to help determine the user's mental state. An example classification model can be a support vector machine classification model. Another example classification model can be a shrinkage linear discriminant analysis model. The determination can be processed and/or presented to a user via interface application 1630 or transmitted to an external system (or entities 1750), for example, a device or system accessible by a caregiver or researcher.

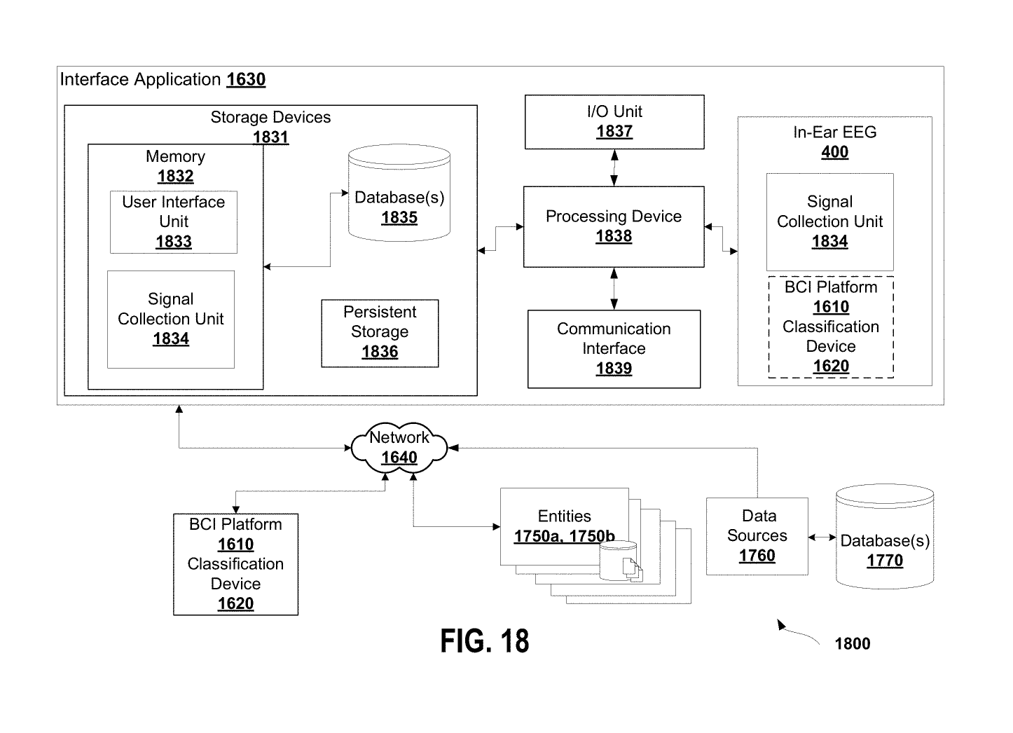

FIG. 18 is a view of an example interface application 1630, in accordance with some embodiments. In some embodiments, interface application 1630 includes a classification device 1620. In some embodiments, interface application 1630 is connected to a headset associated with or housing a BCI platform 1610 and classification device 1620. The headset may include multiple electrodes 102 to collect EEG data when connected to a user's scalp. In some embodiments, the headset may comprise the in-ear EEG device 400. The signals may be collected by signal collection unit 1834, which may connect to BCI platform 1610 optionally housed within the headset. The BCI platform 1610 can create and/or use one or more classifiers as described above. For example, the BCI platform 1610 within a headset or in-ear EEG device 400 can train and retrain a classifier using EEG data from one or more sessions from a single user engaged with interface application 1630 or headset or in-ear EEG device 400. BCI platform 1610 can use the classifier to classify mental states of the user using further EEG signals. BCI platform 1610 may be operable as described above.

In some embodiments, signal collection unit 1834 may be associated with an interface application 1630 that does not include a headset or in-ear EEG device 400. Signal collection unit 1834 can gather data, for example EEG data, from a user engaged with interface application 1630. Interface application 1630 can then cause transmission of data, the EEG signals, processed data or processed EEG signals, or other information to a BCI platform 1610 and/or classification device 1620 over a network 1640 (or multiple networks). The BCI platform 1610 can train and retrain a classifier using EEG data from one or more sessions from a single user engaged with interface application 1630 or headset or in-ear EEG device 400. BCI platform 1610 can use the classifier to classify mental states of the user using further EEG signals. BCI platform 1610 may be operable as described above.

In some embodiments, interface application 1630 connects to a BCI platform 1610 and classification device 1620 over a network 1640 (or multiple networks).

Each I/O unit 1837 enables the interface application 1630 (including headset or in-ear device 400) to interconnect with one or more input devices, such as a keyboard, mouse, camera, touch screen, microphone, electrodes, headset, or other sensory collection devices, for example, that can detect brain activity or mental state. Each I/O unit 1837 also enables the interface application 1630 (including headset or in-ear EEG device 400) to interconnect with one or more output devices such as a display screen, speaker, or other devices presenting visuals, haptics, or audio.

A processing device 1838 can execute instructions in memory 1832 to configure user interface unit 1833 and signal collection unit 1834. A processing device 1838 can be, for example, any type of general-purpose microprocessor or microcontroller, a digital signal processing (DSP) processor, an integrated circuit, a field programmable gate array (FPGA), a reconfigurable processor, or any combination thereof.

Memory 1832 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Storage devices 1831 can include memory 1832, databases 1835, and persistent storage 1836.

Each communication interface 1839 can enable the interface application 1630 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g., Wi-Fi, WiMAX), SS7 signaling network, fixed line, local area network, wide area network, and others, including any combination of these.

The interface application 1630 can be operable to register and authenticate users (using a login, unique identifier, and password for example) prior to providing access to applications, a local network, network resources, other networks and network security devices. The BCI platform 1610 may serve one user or multiple users.

The database 1835 may be configured to store information associated with or created by the classification device 1620. Database 1835 and/or persistent storage 1836 may be provided using various types of storage technologies, such as solid state drives, hard disk drives, flash memory, and may be stored in various formats, such as relational databases, non-relational databases, flat files, spreadsheets, extended markup files, and so on.

User interface unit 1833 can manage the dynamic presentation, receipt, and manipulation of data, such as for example, input received from interface application 1630. User interface unit 1833 can associate the mental state of the user, for example, gathered by a signal collection unit 1834 and classified by a BCI platform 1610, as a mental state and cause storage of same in storage devices 1831 or transmission of same over network 1640 (or multiple networks). As another example, user interface unit 1833 can facilitate validation of a user mental state with the result determined by a BCI platform 1610 or classifier. The interface application 1630 can gather the mental state via I/O unit 1837 connected to a keyboard, touchscreen, mouse, microphone, or other sensory device. User interface unit 1833 can associate the mental state with the result determined by a BCI platform 1610 or classifier to verify the accuracy of the BCI platform 1610 or classifier. In some embodiments, interface application 1630 can transmit the response to a BCI platform 1610.

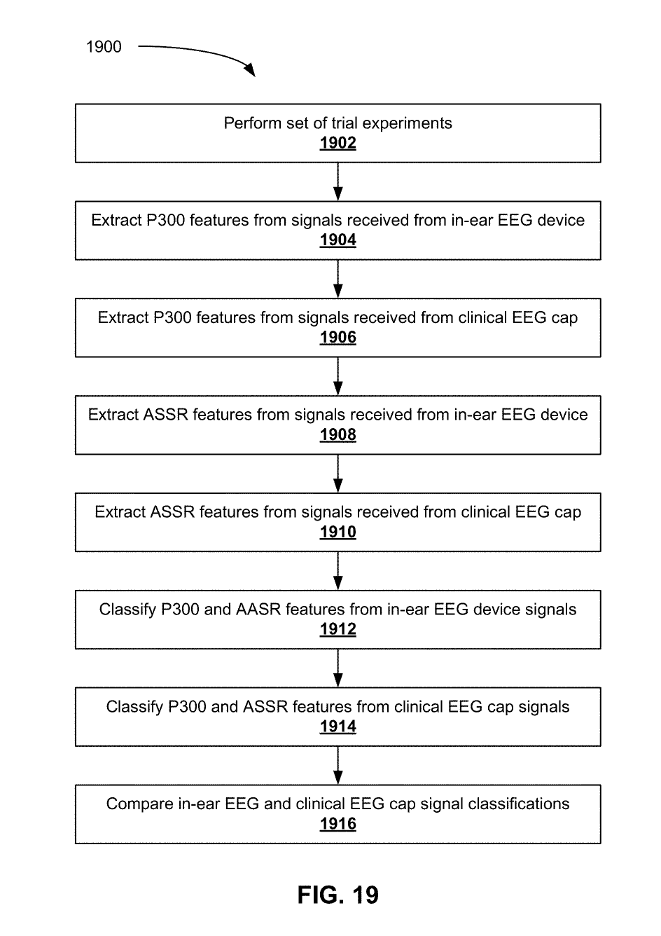

FIG. 19 illustrates, in a flowchart, an example of a method 1900 of validating an in-ear EEG device 400 for use as a BCI, in accordance with some embodiments. The method begins with performing 1902 a set of trial experiments on a plurality of subjects, where each subject is wearing the in-ear EEG device 400 and a clinical EEG cap. Next, P300 features are extracted 1904 from signals received from the in-ear EEG device 400. Next, P300 features are extracted 1906 from signals received from the clinical EEG cap. It is understood that step 1906 may be performed before step 1904. Next, auditory steady-state response (ASSR) features are extracted 1908 from the in-ear EEG device 400. Next, AASR features are extracted 1910 from the clinical EEG cap. It is understood that step 1910 may be performed before step 1908. Next, the P300 features and ASSR features received from the in-ear EEG device 400 signals are classified 1912. Next, the P300 features and ASSR features received from the clinical EEG cap signals are classified 1914. It is understood that step 1914 may be performed before step 1912. Next, the in-ear EEG classifications are compared 1916 with the clinical EEG cap signal classifications. Other steps may be added to the method 1900.

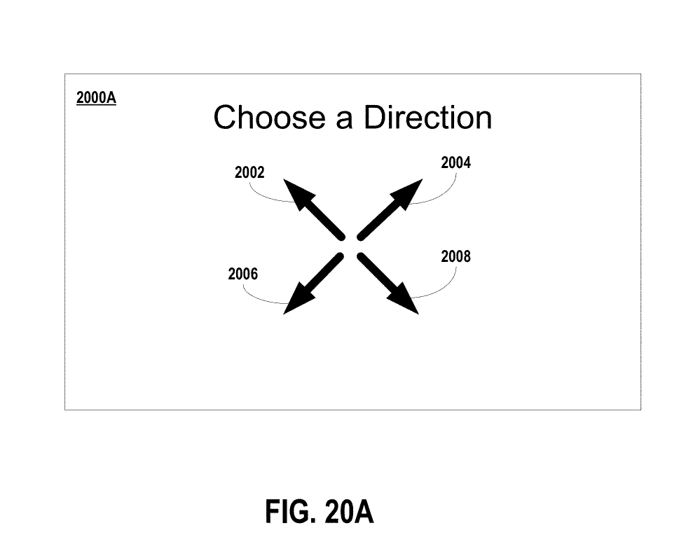

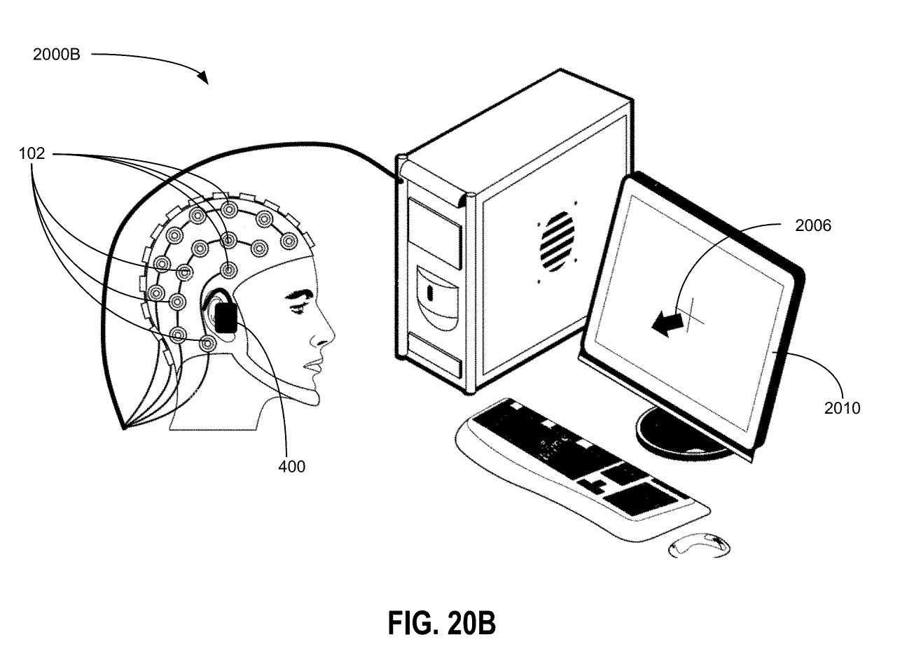

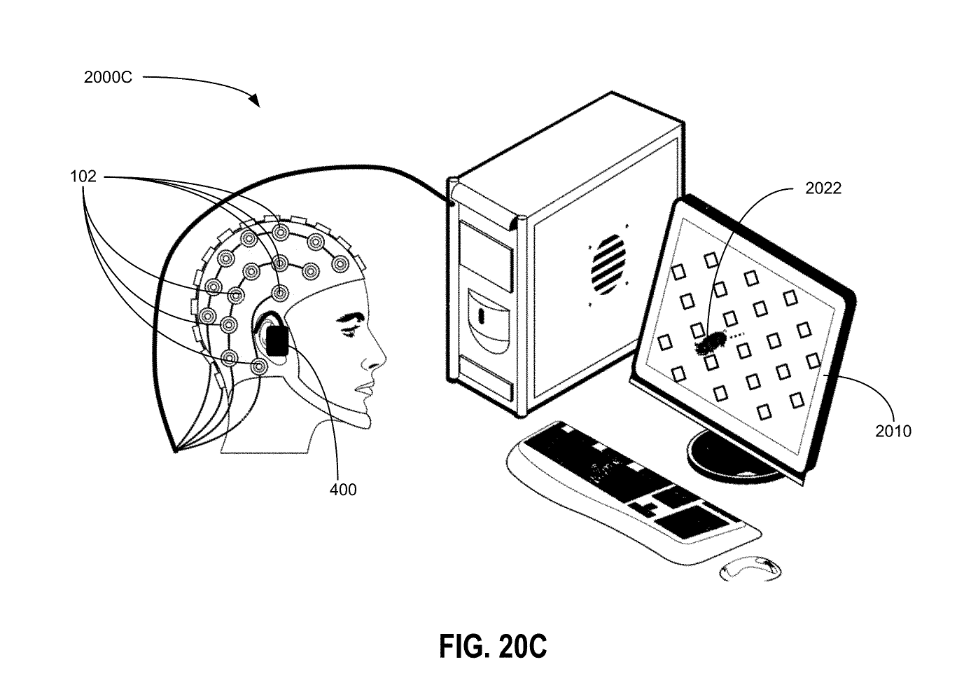

An EEG device and BCI system may be used for visual spatial imagery tasks. For example, visual cues may be displayed on an output unit to a participant. FIG. 20A illustrates, in a display, an example of four directional visual cues 2000A, in accordance with some embodiments. The visual cues comprise an upper-left arrow 2002, an upper right arrow 2004, a lower left arrow 2006 and a lower right arrow 2008. The participant is instructed to choose a direction. A visual cue is presented to the participant. If the visual cue does not match the direction they chose, then the participant is to rest which causes the presentation of another visual cue. If the visual cue does match the direction they chose, then the participant is to visualize the movement of a character in a game.

FIG. 20B illustrates an example of a system environment 2000B showing a participant wearing electrode sensors 102 (i.e., electrodes 102) watching a display unit 2010, in accordance with some embodiments. The output (i.e., display unit 2010) is displaying a visual cue for the direction lower-left 2006. The participate is to visualize a movement of a character in that direction. In some embodiments, the electrode sensors 102 may comprise electrodes 102 on an earpiece 402 of an in-ear EEG device 400.

FIG. 20C illustrates another example of a system environment 2000C showing the participant wearing the electrode sensors 102 watching the display unit 2010, in accordance with some embodiments. Here, the character 2022 is correctly moving in the lower-left direction in response to the participant's visualization. In this example, the brain-state that the participant would experience during visualization would be detected by one or more sensors 102 (e.g., electrodes 102). In some embodiments, the electrode sensors 102 may comprise electrodes 102 on an earpiece 402 of an in-ear EEG device 400.

The EEG signals received by the electrodes 102 may be pre-processed by a collector device and sent to an acquisition unit in a server. The EEG data may then be sent to a processor to determine the visual imagery of the participant. A presentation unit may receive the brain-state and generate the visual elements of the character 2022 moving along the lower-left direction. The display controller issues control commands to the display device 2010 to update the interface with the visual elements (e.g., have the character 2022 move along the lower-left direction.

The example described in FIGS. 20A to 20C involved the use of active BCI monitoring. However, passive BCI monitoring can be applied in parallel to detect the brain-state that the participant would experience during performance of the mental task. For example, the participant may experience frustration if the task is not successful. Such mental state or brain activity would be detected by one or more sensors 102 (e.g., electrodes 102).

The foregoing discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

Although the embodiments have been described in detail, it should be understood that various changes, substitutions and alterations can be made herein.

Moreover, the scope of the present application is not intended to be limited to the particular embodiments of the process, machine, manufacture, composition of matter, means, methods and steps described in the specification.

As can be understood, the examples described above and illustrated are intended to be exemplary only.

What is claimed is:

1. An in-ear electroencephalography (EEG) device comprising:
an enclosure having a power switch, analog output, power input, and processor, the processor configured to receive EEG data and generate output data for the analog output;
an earpiece having two electrodes to collect the EEG data, the earpiece coupled to the enclosure to transmit the EEG data to the processor; and
an over-ear support arm having a reference electrode to collect the EEG data, the over-ear support arm coupled to an enclosure to transmit the EEG data to the processor.

2. An in-ear electroencephalography (EEG) device comprising:
an over-ear support arm coupled to an enclosure, the enclosure comprising a printed circuit board (PCB) of the device and including:
an analog output;
a power input;
a processor configured to receive EEG data and generate output data; and
a header for connecting an earpiece to the EEG device.

3. The in-ear EEG device as claimed in claim 2, further comprising the earpiece connected to the header.

4. The in-ear EEG device as claimed in claim 3, wherein the header is a two-pin header on the PCB.

5. The in-ear EEG device as claimed in claim 3, further comprising two electrodes on the earpiece and a reference electrode on the over-ear support arm.

6. The in-ear EEG device as claimed in claim 5, wherein:
the reference electrode follows a curve of the over-ear support arm; and
the two electrodes on the earpiece serve as positive and negative inputs.

7. The in-ear EEG device as claimed in claim 2, further comprising a power switch.

8. The in-ear EEG device as claimed in claim 2, further comprising a notch filter on the PCB.

9. The in-ear EEG device as claimed in claim 8, wherein the notch filter is centered at 60-Hz.

10. The in-ear EEG device as claimed in claim 2, further comprising a high-pass filter on the PCB.

11. The in-ear EEG device as claimed in claim 10, wherein the high-pass filter is a 1-Hz high-pass filter for removing 0-Hz (DC) offset in a signal.

12. The in-ear EEG device as claimed in claim 2, further comprising a low-pass filter on the PCB.

13. The in-ear EEG device as claimed in claim 12, wherein the low-pass filter is a 100-Hz low-pass filter.

14. The in-ear EEG device as claimed in claim 2, further comprising:
a 60-Hz notch filter;
a 1-Hz high-pass filter; and
a 100-Hz low-pass filter;
wherein a signal input is passed by a first gain stage, the notch-filter, the high-pass filter, a second gain stage and the low-pass filter.

15. The in-ear EEG device as claimed in claim 2, further comprising:
a first 1-Hz high-pass filter;
a second 1-Hz high-pass filter; and
a 40-Hz low-pass filter;
wherein a signal input is passed by a first gain stage, the first high-pass filter, a second gain stage, the second high-pass filter and the low-pass filter.

16. The in-ear EEG device as claimed in claim 2, further comprising:
a buffer for storing EEG signals; and
an amplifier for increasing the signal amplitude.

17. A method of validating an in-ear electroencephalography (EEG) device for use as a brain-computer interface (BCI), the method comprising:
performing a set of trial experiments on a plurality of subjects, each subject wearing the in-ear EEG device and a clinical EEG cap;
extracting P300 features from signals received from the in-ear EEG device;
extracting P300 features from signals received from the clinical EEG cap;
extracting auditory steady-state response (ASSR) features from the signals received from the in-ear EEG device;
extracting ASSR features from the signals received from the clinical EEG cap;
classifying the P300 features and ASSR features received from the in-ear EEG device signals;
classifying the P300 features and ASSR features received from the clinical EEG cap signals; and
comparing the in-ear EEG classifications and the clinical EEG cap signal classifications.

18. A non-transitory computer-readable storage medium comprising computer-executable instructions for validating an in-ear electroencephalography (EEG) device for use as a brain-computer interface (BCI), the computer-executable instructions causing a processor to:
extract P300 features from signals received from an in-ear EEG device;
extract P300 features from signals received from a clinical EEG cap;
extract auditory steady-state response (ASSR) features from the signals received from the in-ear EEG device;
extract ASSR features from the signals received from the clinical EEG cap;
classify the P300 features and ASSR features received from the in-ear EEG device signals;
classify the P300 features and ASSR features received from the clinical EEG cap signals; and
compare the in-ear EEG classifications and the clinical EEG cap signal classifications.
